(19) United States
(12) Patent Application Publication
(10) Pub. No.: US 2010/0048686 A1
(43) Pub. Date: Feb. 25, 2010

(54) Perfume Composition For Temperature Sense Control, Sense Control Article, Sense Control Method, And Perfume Map

(75) Inventors: Ken Shoji, Yokohama-shi (JP); Sumie Taguchi, Yokohama-shi (JP); Yushi Terajima, Yokohama-shi (JP)

Correspondence Address: RANKIN, HILL & CLARK LLP, 23755 Lorain Road - Suite 200, North Olmsted, OH, 44070-2224, US

(73) Assignee: SHISEIDO CO., LTD., Tokyo (JP)

(21) Appl. No.: 12/545,914

(22) Filed: Aug. 24, 2009

Related U.S. Application Data
(63) Continuation of application No. 10/570,634, filed on Mar. 3, 2006, now abandoned.

(30) Foreign Application Priority Data
Sep. 5, 2003 (JP) 2003-314742

(51) Int. Cl. A61K 8/49; A61K 8/00; A61K 8/34; A61K 8/35; A61Q 13/00

(52) U.S. Cl. 514/450; 514/456; 514/701; 514/465; 514/449; 514/690; 514/468; 514/732

(57) ABSTRACT

It is an object of the present invention to discover that a perfume can change a person's temperature sense and humidity sense, to identify a perfume component that can change temperature sense, and to provide a perfume composition for controlling temperature sense that contains this perfume component, and to a sense control method or sense control article such as an aromatic or a cosmetic that is beneficial for controlling temperature sense, and to a perfume map for selecting a perfume suited to controlling temperature sense. The perfume composition is characterized in that a perfume component that changes the temperature subjectively sensed by a person (temperature sense) is contained, and that the perfume component that changes the temperature sense is a perfume component identified on the basis of a temperature image obtained from test subject assessment.

### TECHNICAL FIELD

The present invention relates to a perfume composition for temperature sense control, which changes the subjective temperature sense of a person by means of aroma, and to a temperature sense control article that contains this perfume composition, to a temperature sense control method, and to a

### BACKGROUND ART

Colors have long been known to affect people's temperature sense, time sense, weight sense, and sense of size. For instance, warm colors such as red, orange, and yellow project a warm or expansive image, and have the effect of changing a person's senses in that direction, whereas cool colors such as black, blue, white, and purple are known to project a cool or contracting image. Meanwhile, it is known that fragrances project sense images such as sweet, thick, transparent, or bracing, and that fragrances can arouse emotions, impart a pleasurable feeling, relieve stress, improve work efficiency, and have other such effects. For example, Japanese Laid-Open Patent Application 2001-49286 discloses an invention related to a perfume component that eases stress caused by lack of sleep. Nevertheless, there has been no research into the relationship between aroma and temperature sense, and it has never once been proposed up to now that temperature sense could be changed by aroma.

Patent Document: Japanese Laid-Open Patent Application

### DISCLOSURE OF THE INVENTION

It is an object of the present invention to discover that a perfume can change a person's temperature sense, to identify a perfume component that can change temperature sense, and to provide a perfume composition for controlling temperature sense that contains this perfume component, and to a sense control method or sense control article such as an aromatic or a cosmetic that is beneficial for controlling temperature sense, and to a perfume map or a method for selecting a perfume suited to controlling temperature sense.

The first invention is a perfume composition for temperature sense control, containing a perfume component that changes the temperature subjectively sensed by a person (temperature sense), wherein this composition contains a perfume component that raises the temperature subjectively sensed by a person (temperature sense), or a perfume component that lowers the temperature subjectively sensed by a person (temperature sense).

The perfume component that changes the temperature sense is a perfume component identified on the basis of a temperature image obtained from test subject assessment, and the scent of the overall perfume composition is [that of] a perfume composition identified on the basis of a temperature image obtained from a test subject.

The second invention is an article for controlling temperature sense and/or usability/skin feel, containing the above-mentioned perfume composition and used to change the temperature sense and/or usability/skin feel of an article.[¹] An example is a cosmetic for controlling temperature sense and/or usability/skin feel, which changes the temperature sense and/or usability/skin feel of a cosmetic.

The third invention is a method for controlling temperature sense and/or usability/skin feel, wherein temperature sense and/or usability/skin feel is changed by having a person smell the above-mentioned perfume component. An example is when the temperature sense and/or usability/skin feel [experienced by] a person within a specific space is changed by releasing the perfume composition within this space.

The fourth invention is a method in which an image of the aroma perceived when a person smells a perfume component and/or perfume composition is obtained by positioning a specific perfume component and/or perfume composition, on the basis of the aroma image, on a coordinate plane (map) having X and Y axes, with the X axis having “tender” (mild, sweet) and “sharp” (bracing) at opposite ends, and the Y axis having “natural” (transparent, bright) and “rich” (thick, sultry) at opposite ends, and [how much] the temperature sense and/or usability/skin feel is changed by this perfume component and/or perfume composition is estimated from the position on the map. The fifth invention is a perfume map with which an image of the aroma perceived when a person smells a perfume component and/or perfume composition is obtained by positioning a specific perfume component and/or perfume composition, on the basis of the aroma image, on a coordinate plane (map) having X and Y axes, with the X axis having “tender” (mild, sweet) and “sharp” (bracing) at opposite ends, and the Y axis having “natural” (transparent, bright) and “rich” (thick, sultry) at opposite ends, and [how much] the temperature sense and/or usability/skin feel is changed by this perfume component and/or perfume composition is estimated from the position on the map.

The perfume component that raises the temperature sense is a component selected from the group composed of vanillin, 4-tert-butyl-α-methylhydrocinnamic aldehyde, heliotropine, 4,6,6,7,8,8-hexamethyl-1,3,4,6,7,8-hexahydrocyclopentabenzopyran, γ-undecalactone, β-ionone, cumin oil, lavender oil, clove oil, 3α,6,6,9α-tetramethyldodecahydronaphtho[2,1-b]furan, and maltol.

The perfume component that lowers the temperature sense is a component selected from the group composed of peppermint oil, bergamot oil, spearmint oil, lime oil, 7-methyl-3,4-dihydro-(2H)-1,5-benzodioxepin-3-one, chamomile oil, 2,4-dimethyl-3-cyclohexenyl carboxyaldehyde, majolaine oil, patchouli oil, jasmine absolute, sandalwood oil, geranium oil, rose oil, and methyl-N-3,7-dimethyl-7-hydroxyoctylidene-anthranilate.

The sixth invention is a method wherein the moisture content of skin is increased by having a person smell the a above-mentioned perfume component or perfume composition that raises the temperature sense, or the sebum content of skin is reduced by having a person smell the above-mentioned perfume component or perfume composition that lowers the temperature sense.

With the present invention, it is possible to identify a perfume component and perfume composition that can change temperature sense, and to change and control temperature sense by means of an aroma given off by a perfume component. Also, it is possible to easily and reliably select a perfume component and perfume composition capable of changing temperature sense on the basis of a temperature image obtained from a test subject. Also, it is possible to change and control the temperature sense and/or usability/skin feel [experienced by] a person in a space in which the user or aroma is present, by using an article, cosmetic, or the like containing the perfume composition for temperature sense control of the present invention. Furthermore, it is possible to estimate the temperature sense and/or usability/skin feel that can be changed by a perfume component and/or perfume composition by utilizing a perfume map.

### BRIEF DESCRIPTION OF THE DRAWINGS

FIG. 1 is a diagram illustrating a method for verifying the change in temperature sense brought about by aroma with a cream;

FIG. 2 is a graph of the relationship between cream temperature sense brought about by aroma and the “freshness[²] of the skin”;

FIG. 3 is a graph of the relationship between cream temperature sense brought about by aroma and the “moistness of the skin”;

FIG. 4 is a diagram of a perfume map;

FIG. 5 is a graph of the relationship between aroma and space temperature sense;

FIG. 6 is a conceptual diagram of an experimental apparatus for confirming the difference in temperature sense brought about by aroma within a space;

FIG. 7 is a graph of the assessment of temperature sense in this experimental apparatus; and

FIG. 8 is a graph of the assessment of the intensity of aroma in this experimental apparatus.

### BEST MODE FOR CARRYING OUT THE INVENTION

Preferred embodiments of this invention will now be described in detail. This invention identifies a perfume component that can change a person's temperature sense by aroma, and provides a perfume composition for controlling temperature sense that contains this perfume component, and also involves preparing an article for controlling temperature sense that contains this perfume composition, and to controlling temperature sense by volatilizing this perfume composition. Also, it involves utilizing a perfume map to allow the desired perfume for controlling temperature sense to be selected easily and reliably.

As a result of various research, the inventors discovered that the aroma given off by a specific perfume component can change the temperature subjectively sensed by a person (temperature sense). 12 test subjects put there hands into water held at a constant temperature, and the subjectively sensed temperature of the water was assessed on a seven-point scale in which 7 was hot, 6 was warm, 5 was somewhat warm, 4 was “can't say,” 3 was somewhat cold, 2 was cold, and 1 was extremely cold. The same 12 test subjects were asked to smell a peppermint aroma while once again putting their hands into water held at a constant temperature, and the subjectively sensed temperature of the water was assessed on a seven-point scale. The same experiment was repeated by raising the water temperature one degree at a time, and the testers looked for the temperature at which the assessment given under conditions of smelling the peppermint aroma was the same as the assessment given under conditions of no aroma at 28° C. As a result, the average value for the assessment when hands were put in water adjusted to 28° C. was 4.8 (“somewhat warm”) under conditions of no aroma, and 3 (“somewhat cold”) under conditions of smelling the aroma. When the water temperature was raised to 32° C., the assessment was 4.7 (“somewhat warm”) under conditions of smelling the aroma, and it was found that the assessment of 4.8 (“somewhat warm”) with 28° C. water under conditions of no aroma was substantially the same as the assessment with 32° C. water under conditions of smelling peppermint. Specifically, the aroma of peppermint was found to change the temperature sense by approximately 4° C.

Also, to examine the change in temperature sense with and without aroma, the 12 test subjects put their hands in water with a temperature of 28° C., and the subjectively sensed temperature was assessed on a seven-point scale in the same manner as above. When there was no aroma, the assessment was 5.25 (“somewhat warm”), but under conditions of smelling the aroma of L-carvone, which is the main component of spearmint, the assessment was 4 (“can't say”). Further, the same experiment was conducted under conditions of smelling the aroma of L-menthol, which is the main component of peppermint. Under conditions of no aroma, the assessment was 4.63 (“somewhat warm”), and with an aroma, the assessment was 4.125 (“can't say”). These experiments revealed that even with the same 28° C. water, how warm the water felt under conditions of no aroma differed slightly with the experiment.

Next, to identify the perfume components capable of changing the temperature sense of a person for the 24 typical types of perfume discussed below, a plurality of test subjects were asked to smell specific aromas and assess the temperature image of the aroma on a seven-point scale in which 7 was very warm, 6 was warm, 5 was somewhat warm, 4 was “can't say,” 3 was somewhat cold, 2 was cold, and 1 was very cold. A survey was taken by selecting 17 or 18 women 20 to 24 years of age for each perfume component of the 24 types of raw material perfume, strips of odor paper were sprayed with the raw material perfumes, and these were given to the survey respondents.

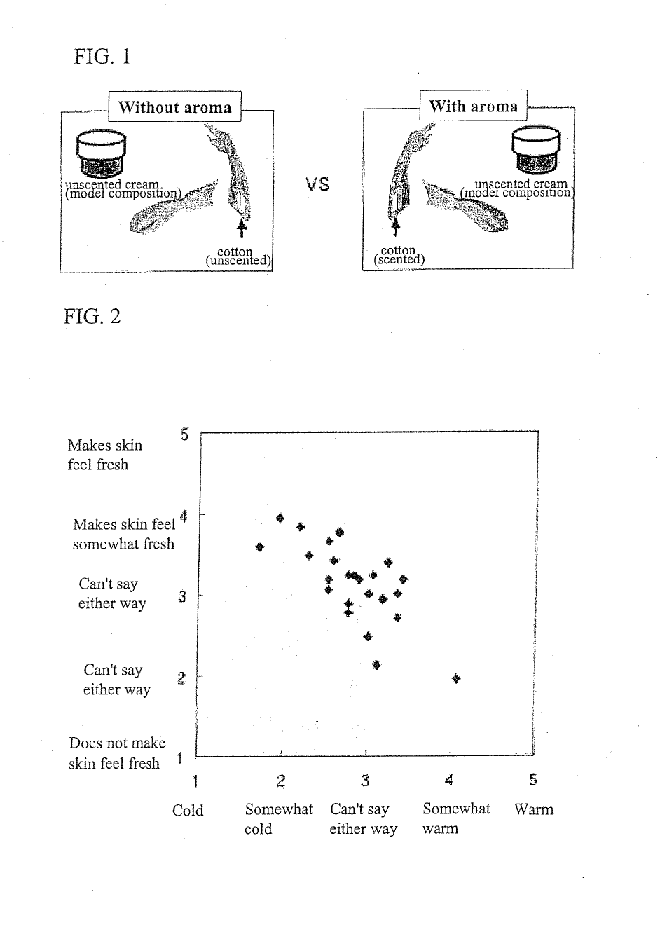

As shown in FIG. 1, the temperature sense for a cosmetic brought about by aroma was compared with the usability/skin feel with and without an aroma, for the 24 typical types of perfume discussed below, and an assessment was made versus no aroma. Just as with the survey conducted for temperature sense above, 17 or 18 women 20 to 24 years of age were selected for this test, cotton to which no perfume had been applied was affixed to one arm, and cotton to which perfume had been applied was affixed to the other arm, the arms were each coated with an unscented cream while the subjected smelled the aroma of the cotton, and the subjectively sensed temperature of the cream was assessed on a five-point scale in which 5 was warm, 4 was somewhat warm, 3 was “can't say,” 2 was somewhat cold, and 1 was cold.

The 24 types of raw material perfume were vanillin, 4-tert-butyl-α-methylhydrocinnamic aldehyde, heliotropine, 4,6,6,7,8,8-hexamethyl-1,3,4,6,7,8-hexahydrocyclopentabenzopyran, γ-undecalactone, β-ionone, cumin oil, lavender oil, clove oil, 3α,6,6,9α-tetramethyldodecahydronaphtho[2,1-b]furan, peppermint oil, bergamot oil, spearmint oil, lime oil, 7-methyl-3,4-dihydro-(2H)-1,5-benzodioxepin-3-one, chamomile oil, 2,4-dimethyl-3-cyclohexenyl carboxyaldehyde, majolaine oil, patchouli oil, jasmine absolute, sandalwood oil, geranium oil, rose oil, and methyl-N-3,7-dimethyl-7-hydroxyoctylidene-anthranilate.

Factor analysis was performed on the assessment results obtained from a plurality of test subjects in the above experiments, and the result was that there is a positive correlation between the temperature image (warm) of an aroma and the subjectively sensed temperature (warm), and the correlation coefficient was 0.67. Thus, it is possible to easily and reliably select a perfume component and a perfume composition capable of changing temperature sense on the basis of the temperature image obtained from a plurality of test subjects as above. Furthermore, the effect of the aroma is the same not only for the perfume component, but for the fragrance of the perfume composition as a whole.

The above experiments also revealed that perfume raw materials having a warm temperature image, and raw material perfumes capable of raising temperature sense were vanillin, 4-tert-butyl-a-methylhydrocinnamic aldehyde, heliotropine, 4,6,6,7,8,8-hexamethyl-1,3,4,6,7,8-hexahydrocyclopentabenzopyran, γ-undecalactone, β-ionone, cumin oil, lavender oil, clove oil, 3α,6,6,9α-tetramethyldodecahydronaphtho[2,1-b]furan, and maltol. A syrup perfume, chocolate perfume and coconut perfume also can raise temperature sense.

Conversely, perfume raw materials having a cool temperature image, and raw material perfumes capable of lowering temperature sense were peppermint oil, bergamot oil, spearmint oil, lime oil, 7-methyl-3,4-dihydro-(2H)-1,5-benzodioxepin-3-one, chamomile oil, 2,4-dimethyl-3-cyclohexenyl carboxyaldehyde, majolaine oil, patchouli oil, jasmine absolute, sandalwood oil, geranium oil, rose oil, and methyl-N-3,7-dimethyl-7-hydroxyoctylidene-anthranilate. Furthermore, the effect of the aroma is the same not only for the perfume component, but for the fragrance of the perfume composition as a whole.

These 24 different perfume raw materials are nothing more than examples, and the present invention is not limited to or by these 24 types. The present invention identifies a perfume component that can change temperature sense, and is designed to be used as a perfume composition that contains this perfume component and is for controlling the temperature sense of a person, and even with other perfume raw materials besides the 24 types discussed above, the present invention encompasses perfume components that can change temperature sense and are identified on the basis of a temperature image obtained from test subject assessment. Also, the perfume map discussed below allows a person's impression of the aroma of a perfume to be utilized to evaluate the temperature sense, as well as the usability/skin feel, of perfume raw materials whose temperature sense is unknown, and the present invention also encompasses perfume raw materials thus evaluated and identified.

Next, the correlation of aroma-induced cosmetic temperature sense, usability, and how the cosmetic actually felt on the skin, and the correlation with the image of the aroma were tested for the above-mentioned 24 different raw material perfumes. As shown in FIG. 1, the test method for a cream involved comparing the aroma-induced temperature sense, usage feel of the cream, and skin feel with those obtained without any aroma. The aroma image was assessed on a seven-point scale from 0 to 6, while the usability/skin feel was assessed on a five-point scale from 5 (“I agree”) to 1 (“I disagree”). In addition to assessing usability, the impression of the aroma of the perfume was also assessed at the same time. To prevent the perfume from modifying the cream, a method was employed in which the perfume component was not directly added to the cream, but was instead applied to the cotton and smelled.

The following wording was selected for usability/skin feel.
How well a cream spreads: light to heavy
Absorption into the skin: fast to slow
Makes the skin look youthful?: yes to no
Makes the skin feel sticky?: yes to no
Makes the skin feel fresh?: yes to no
Makes the skin feel moist?: yes to no
Makes the skin feel non-sticky?: yes to no
Makes the skin feel smooth?: yes to no
Makes the skin feel supple?: yes to no
Makes the skin feel tight?: yes to no
Each of these was assessed on a five-point-scale.
For impressions of aroma, words that readily express the characteristics of an aroma, and four different words (natural, rich, tender, and sharp) were selected as generic terms from among sensory adjectives and emotional adjectives used to express the five senses. Each generic term encompasses specific aroma impression words. For example, “natural” encompasses impression expressions such as transparent, bright, natural, [3] pure, etc.; “rich” encompasses thick, sultry, mature, glossy, sexy, etc.; “tender” encompasses mild, sweet, gentle, graceful, etc; and “sharp” encompasses bracing, sharp, cool, etc. The impression of these words for aromas were evaluated on a seven-point scale from 0 (do not feel at all) to 6 (strongly feel). However, the words used to express usability/skin feel and the words used to express impressions of aroma are nothing more than examples, and [the present invention] is not limited to or by these words. In particular, the aroma assessment terms disclosed in Japanese Laid-Open Patent Application 2001-174450 can be employed as words for expressing aroma impressions.

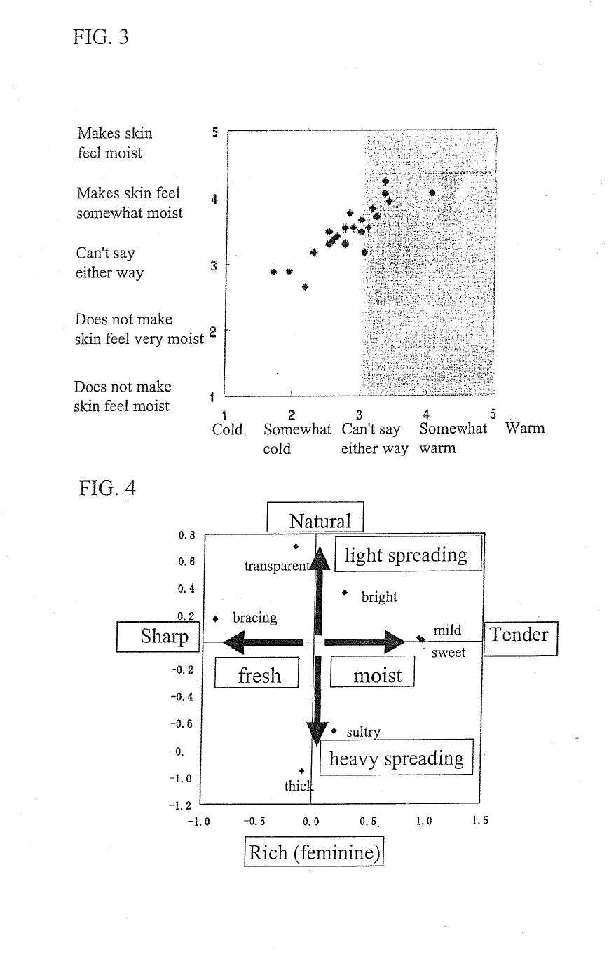

Table 1 gives average values for the assessment of aroma images and usability/skin feel, and the change in the temperature sense of a cream produced by aroma, for eight types of typical perfume raw material FIG. 2 is a graph of the relationship between the change in cream temperature sense brought about by aroma and the “freshness of the skin.” FIG. 3 is a graph of the relationship between temperature sense and the “moistness of the skin.” It can be seen from Table 1 and FIGS. 2 and 3 that a cream having an aroma that is sensed to be cold gives a greater sense of skin freshness, non-stickiness, and tightness than an unscented cream, while a cream having an aroma that is sensed to be warm gives a greater sense of skin moistness and smoothness than an unscented cream. Thus, it will be understood that a cosmetic containing a perfume component capable of controlling temperature sense will be able to change the usability/skin feel by its aroma, so it is possible to control the usability/skin feel of a cosmetic by means of aroma. In Table 1, A is peppermint, B is 4-tert-butyl-α-methylhydroxycinnamic aldehyde, C is vanillin, D is majolaine, E is 2,4-dimethyl-3-cyclohexenyl carboxyaldehyde, F is cumin, G is γ-undecalactone, and H is lime.

TABLE 1

| | Aroma image words | | | | | | |
|---|---|---|---|---|---|---|---|
| | Transparent | Bright | Thick | Sultry | Mild | Sweet | Bracing |
| A | 3.6 | 3.6 | 2.5 | 2.1 | 1.8 | 1.9 | 4.6 |
| B | 3.8 | 3.8 | 2.2 | 2.1 | 3.6 | 3.6 | 2.8 |
| C | 2.0 | 3.6 | 3.8 | 3.2 | 4.5 | 4.9 | 1.2 |
| D | 1.9 | 2.6 | 3.7 | 2.6 | 1.9 | 1.4 | 4.1 |
| E | 3.2 | 3.1 | 2.1 | 1.6 | 2.5 | 2.6 | 2.9 |
| F | 1.5 | 2.1 | 4.0 | 3.1 | 2.6 | 1.9 | 1.9 |
| G | 2.6 | 4.1 | 2.9 | 2.1 | 3.9 | 4.3 | 1.5 |
| H | 3.4 | 4.2 | 2.8 | 2.1 | 1.9 | 1.7 | 4.5 |

| | Usability/Skin Feel | | | | | | | |
|---|---|---|---|---|---|---|---|---|
| | Cream | | | | Non- | Cream | | |
| | temp. | | | | stickiness | temp. | | |
| | sense | Skin | Skin | Skin | of | sense | Skin | Skin |
| | (cold) | tightness | freshness | suppleness | skin | (warm) | smoothness | moistness |
| A | 4.3 | 4.1 | 3.6 | 3.9 | 3.1 | 1.7 | 3.2 | 2.9 |
| B | 2.6 | 3.3 | 3.2 | 3.4 | 2.5 | 3.4 | 3.8 | 3.9 |
| C | 1.9 | 2.5 | 1.9 | 2.7 | 2.2 | 4.1 | 3.9 | 4.1 |
| D | 3.4 | 3.2 | 3.4 | 3.1 | 2.8 | 2.6 | 3.5 | 3.4 |
| E | 3.5 | 2.4 | 3.1 | 3.4 | 2.6 | 2.5 | 3.8 | 3.3 |
| F | 2.9 | 2.7 | 2.1 | 3.1 | 2.4 | 3.1 | 3.0 | 3.5 |
| G | 2.8 | 2.9 | 3.4 | 3.2 | 3.0 | 3.2 | 3.9 | 3.7 |
| H | 3.7 | 3.6 | 3.5 | 3.6 | 3.2 | 2.3 | 3.4 | 3.2 |

| | Usability/Skin Feel | | | | | | | |
|---|---|---|---|---|---|---|---|---|
| | Cream | Absorption | | Stickiness | Cream | Thickness | Plumpness | Firmness |
| | spreading | into | Youthfulness | of | spreading | of | of | of |
| | (light) | skin | of skin | skin | (heavy) | cream | skin | skin |
| A | 3.5 | 3.7 | 3.7 | 3.0 | 2.5 | 2.3 | 2.3 | 3.0 |
| B | 3.8 | 3.5 | 3.8 | 3.1 | 2.2 | 2.5 | 2.2 | 2.9 |
| C | 2.9 | 3.2 | 3.2 | 2.5 | 3.1 | 2.8 | 2.8 | 3.5 |
| D | 3.5 | 3.3 | 3.2 | 3.2 | 2.5 | 2.7 | 2.8 | 2.8 |
| E | 3.3 | 2.9 | 3.7 | 3.2 | 2.7 | 3.1 | 2.3 | 2.8 |
| F | 2.9 | 2.9 | 2.7 | 2.5 | 3.1 | 3.1 | 3.3 | 3.5 |
| G | 3.8 | 3.9 | 3.9 | 3.1 | 2.2 | 2.1 | 2.1 | 2.9 |
| H | 3.7 | 3.2 | 3.6 | 2.9 | 2.3 | 2.8 | 2.4 | 3.1 |

In Table 2, the temperature sense and usability/skin feel that change with aroma are classified by factor analysis into “moist-fresh (factor 1)” and “spreading-absorption (factor 2)”, and the correlation is looked at by factor load. It can be seen from Table 2 that (i) the moist skin feel and fresh skin feel that change with aroma are at opposite ends of the spectrum, (ii) lightness of spreading and heaviness of spreading are at opposite ends of the spectrum, and (iii) the moist-fresh axis and the spreading lightness-heaviness axis are in a perpendicular relationship.

TABLE 2

| | | | Factor 2 |
|---|---|---|---|
| | | Factor 1 | spreading- |
| | | moist-fresh | absorption |
| 1 | Moistness of skin | 0.914 | 0.114 |
| | Warmth or coolness of | 0.886 | −0.097 |
| | cream | | |
| | Smoothness of skin | 0.800 | 0.367 |
| 2 | How well the cream | −0.087 | 0.899 |
| | spread | | |
| | Youthfulness of skin | −0.021 | 0.713 |
| | Absorption into the skin | −0.055 | 0.702 |
| | Non-stickiness of skin | −0.543 | 0.612 |
| 3 | Stickiness of skin | −0.324 | 0.494 |
| | Freshness of skin | −0.739 | 0.476 |
| | Suppleness of skin | −0.783 | 0.354 |
| | Tightness of skin | −0.774 | 0.457 |
| | Contribution (%) | 40.3 | 28.6 |
| | Cumulative contribution | 40.3 | 68.9 |
| | (%) | | |

Table 3 shows the correlation coefficients for aroma image and the temperature sense and/or usability/skin feel that changes with the aroma. It can be seen from Table 3 that an aroma that makes the skin feel fresh scores high in terms of being bright; transparent, and bracing, and scores low in terms of being mild, sweet, and thick. An aroma that makes the skin feel moist scores high in terms of being mild and sweet, and low in terms of being bracing. An aroma that gives the sense of light spreading and quick absorption scores high in terms of being bright and transparent, and low in terms of being thick.

TABLE 3

| | | Fresh (X axis −) | | | | | | | Light spreading, fast | | | |
|---|---|---|---|---|---|---|---|---|---|---|---|---|
| | | | Non- | | | Moist (X axis +) | | | absorption (Y axis +) | | | |
| | | | stickiness | Skin | | Skin | | | Skin | Cream | Absorption | Skin |
| | | Skin | of | supple- | Skin | mois- | Skin | Cream | youthful- | spread- | into | sticki- |
| | | freshness | skin | ness | tightness | ture | smoothness | warmth | ness | ing | skin | ness |
| Impression | Mild | −0.46 | −0.30 | −0.49 | −0.46 | 0.79 | 0.73 | 0.79 | 0.17 | 0.17 | 0.25 | −0.05 |
| of | Sweet | −0.35 | −0.26 | −0.41 | −0.40 | 0.69 | 0.70 | 0.71 | 0.29 | 0.21 | 0.36 | −0.03 |
| aroma | Bright | 0.43 | 0.53 | 0.41 | 0.48 | 0.02 | 0.33 | −0.08 | 0.69 | 0.73 | 0.57 | 0.47 |
| | Transparent | 0.58 | 0.49 | 0.66 | 0.63 | −0.25 | 0.06 | −0.42 | 0.68 | 0.62 | 0.34 | 0.36 |
| | Bracing | 0.71 | 0.57 | 0.77 | 0.80 | −0.78 | −0.55 | −0.82 | 0.14 | 0.23 | 0.05 | 0.38 |
| | Thick | −0.26 | −0.27 | −0.24 | −0.17 | −0.06 | −0.30 | 0.21 | −0.68 | −0.48 | −0.27 | −0.16 |
| | Sultry | 0.63 | −0.50 | −0.67 | −0.53 | 0.26 | −0.01 | 0.44 | −0.57 | −0.55 | −0.20 | −0.45 |

Since the correlation shown in Tables 1 to 3 and FIGS. 2 and 3 exists between the aroma image and temperature sense and usability/skin feel, we can obtain the coordinate plane (map) shown in FIG. 4. FIG. 4 shows a perfume map with which an image of the aroma perceived when a person smells a perfume component and/or perfume composition is obtained by positioning a specific perfume component and/or perfume composition, on the basis of the aroma image, on a coordinate plane (map) having X and Y axes, with the X axis having “tender” (mild, sweet) and “sharp” (bracing) at opposite ends, and the Y axis having “natural” (transparent, bright) and “rich” (thick, sultry) at opposite ends, and [how much] the temperature sense and/or usability/skin feel is changed by this perfume component and/or perfume composition is estimated from the position on the map.

As described in relation to FIG. 1, since the warm-cold impression of an aroma is positioned on the same axis as temperature sense, the X axis of the perfume map in FIG. 4 is the axis of temperature sense that changes with aroma, with “warm” on the right end and “cold” on the left end. The X axis is also the axis of “fresh-moist” for usability/skin feel, with “moist (skin feels soft, skin is moist)” on the right end, and “fresh (skin feels nicely tight, skin feels fresh, skin feels supple, and skin feels non-sticky)” on the left end.

In contrast, the Y axis is the aroma impression axis, with the top end being “natural” and the bottom end “rich,” and at the same time, the Y axis is the axis of usability/skin feel indicating whether spreading is light or heavy, with the top end being “light spreading (fast absorption into the skin, skin looks youthful, skin is not sticky)”, and the bottom end “heavy spreading (cream is thick, skin is plump, skin is firm).” This Y axis is not correlated to temperature sense.

By assessing the aroma impression of the perfume component or the perfume composition as a whole by the above method, and positioning it on a map such as that shown in FIG. 4, it is possible to determine perfumes that change temperature sense, as well as usability/skin feel including skin smoothness, skin moistness, skin tightness, skin suppleness, and non-stickiness of skin, and the extent of the effect of these perfumes, from the position on the X axis of this map. From the position on the Y axis can be determined perfumes that change usability/skin feel including cream spreading, absorption into the skin, skin youthfulness, skin stickiness, cream thickness, skin plumpness, and skin firmness, and the extent of the effect of these perfumes. Thus, the use of such a map makes it possible to estimate the temperature sense and/or usability/skin feel that can be changed by almost any perfume component or perfume composition, and to easily and reliably select a perfume having the desired effect.

Examples of perfume raw materials that give the perception of the above-mentioned moist usability/skin feel include vanillin, 4-tert-Butyl-α-methylhydrocinnamic aldehyde, heliotropine, 4,6,6,7,8,8-hexamethyl-1,3,4,6,7,8-hexahydrocyclopentabenzopyran, γ-undecalactone, β-ionone, cumin oil, lavender oil, clove oil, and 3 α,6,6,9a-tetramethyldodecahydronaphtho[2,1-b]furan. Examples of perfume raw materials that give the perception of the above-mentioned fresh usability/skin feel include peppermint oil, bergamot oil, spearmint oil, lime oil, 7-methyl-3,4-dihydro-(2H)-1,5-benzodioxepin-3-one, chamomile oil, 2,4-dimethyl-3-cyclohexenyl carboxyaldehyde, majolaine oil, patchouli oil, jasmine absolute, sandalwood oil, geranium oil, rose oil, and methyl-N-3,7-dimethyl-7-hydroroxyoctylidene-anthranilate. These coincide with the above-mentioned perfume raw materials that give a warm temperature sense and a cold temperature sense.

Examples of perfume raw materials that give the perception of heavy-spreading usability/skin feel include cumin oil, patchouli oil, clove oil, jasmine absolute, methyl-N-3,7-dimethyl-7-hydroroxyoctylidene-anthranilate, and vanillin, and examples of perfume raw materials that give the perception of light-spreading usability/skin feel include γ-undecalactone, 4-tert-butyl-α-methylhydrocinnamic aldehyde, heliotropine, rose oil, 7-methyl-3,4-dihydro-(2H)-1,5-benzodioxepin-3-one, lime oil, geranium oil, chamomile oil, bergamot oil, peppermint oil, β-ionone, majolaine oil, 4,6,6,7,8,8-hexamethyl-1,3,4,6,7,8-hexahydrocyclopentabenzopyran, lavender oil, 2,4-dimethyl-3-cyclohexenyl carboxyaldehyde, spearmint oil, and 3α,6,6,9α-tetramethyldodecahydronaphtho[2,1-b]furan.

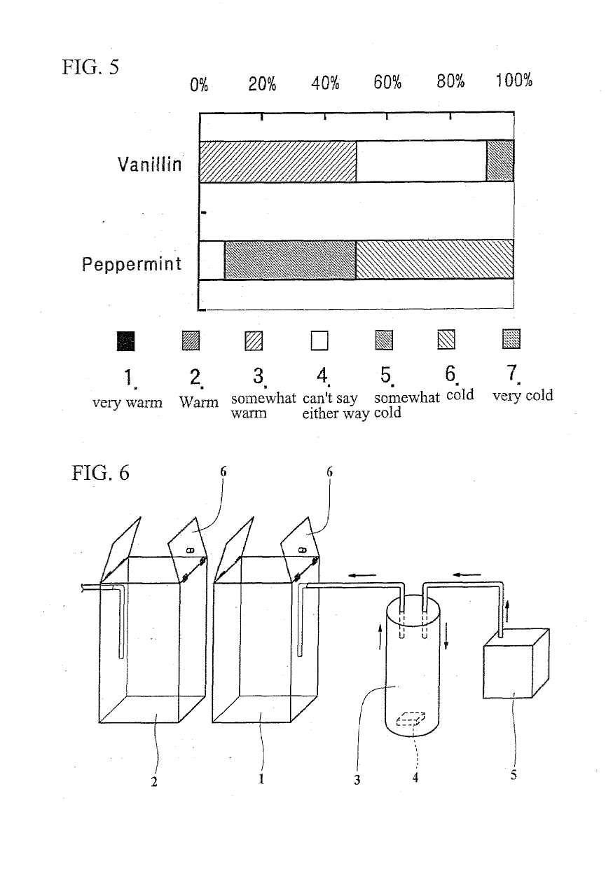

Also, it is possible to change the temperature sense of a person located in a specific space by dispersing in that space, by volatilization or another such means, a perfume composition containing the above-mentioned perfume component capable of changing a person's temperature sense, and having the person smell the aroma. To test this, a clear box containing scented paper and another clear box containing unscented paper were readied, and an experiment was conducting by having a test subject put his or her face into each of the boxes and compare the temperature sense of the space. The difference from no aroma was assessed on a seven-point scale in which 7 was much colder, 6 was colder, 5 was somewhat colder, 4 was “can't say,” 3 was somewhat warmer, 2 was warmer, and 1 was much warmer. 12 women 20 to 24 years of age participated in this experiment, using peppermint and vanillin, at a room temperature of 23 degrees and a humidity of 46%. FIG. 5 is a graph of the assessment results, and it can be seen that the temperature sense of a person located in a space can be changed by aroma. When the perfume composition is dispersed in the space so that the person will smell it, the concentration of the aroma is preferably about 0.01 to 10 ppm. With peppermint oil, for instance, about 0.4 ppm is favorable.

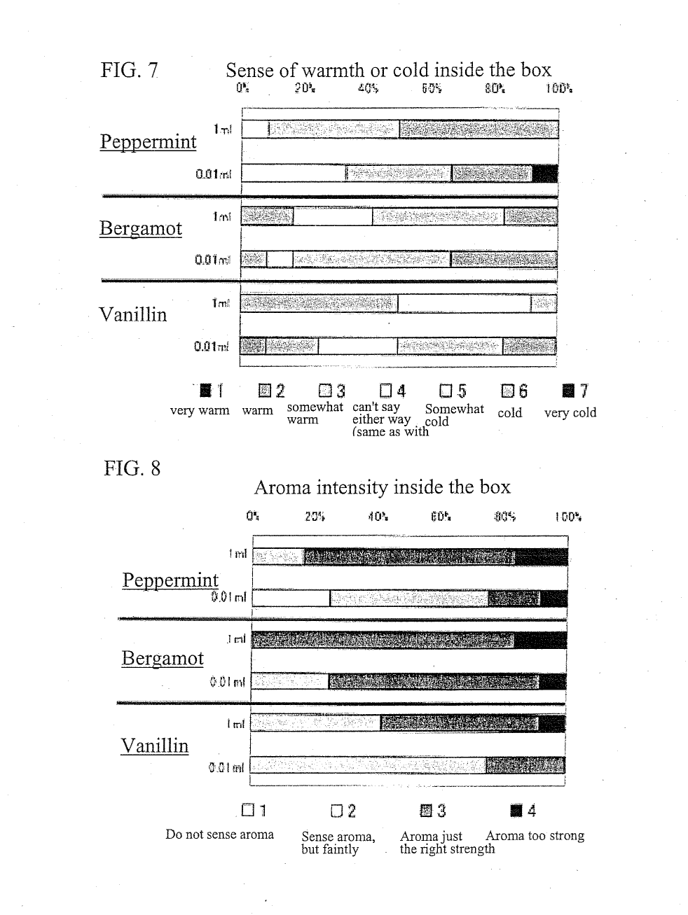

As shown in FIG. 6, two clear boxes 1 and 2 were readied, the perfume component 4 inside a perfume box 3 was pumped by an air pump 5 into one of the boxes [1], and no aroma at all was pumped into the other box 2. The test subject put his or her face into first one box and then the other and compared the temperature sense of the space. This experiment was conducted for three different aromas, namely, peppermint, bergamot, and vanillin. A lid 6 that could be opened and closed was provided to the top of each of the boxes 1 and 2. The test subjects and experiment conditions were the same as discussed above. These results are given in FIGS. 7 and 8. FIG. 7 is a graph of the assessment of the sense of warmth or cold inside the boxes, and shows that the temperature sense perceived by the face changes with the aroma. For example, peppermint gave a cold sensation, and the higher was the concentration, the stronger was the assessment of cold. Vanillin, meanwhile, gave a warm sensation, and the higher was the concentration, the stronger was the assessment of warmth. Bergamot gave a cold sensation, but this cold sensation was assessed as being stronger when the concentration was not too high, and it can be seen that the effect is less likely to be obtained if the concentration of the aroma is too high, and a suitable temperature sense will be obtained at just the right concentration. FIG. 8 is a graph of the assessment of the aroma intensity inside the box, as a function of concentration.

It is also possible to impart to a person inside a specific space a warm or cold sensation that is changed from that of the external space by dispersing a specific aroma into that space by means of a dehumidifier, humidifier, fan, air conditioner, or the like internally equipped with an aromatic liquid container designed to allow the aromatic liquid to be discharged. The means for dispersing the aroma is not limited to the above, and a filter impregnated with an aromatic may be removably attached to the discharge opening of the above-mentioned air conditioner or the like. The cold-sensation perfumes 1 to 4 and warm-sensation perfumes 1 to 4 discussed below are favorable as this aromatic, but the present invention is not limited to these.

The experiment discussed below revealed that with a perfume capable of changing temperature sense, an aroma that raises the temperature sense has the effect of increasing the moisture content of the skin, while an aroma that lowers the temperature sense has the effect of reducing the sebum content of the skin.

### Experimental Methodology

12 female test subjects 20 to 24 years of age were asked to wash their face and attach a piece of scented cotton under their nostrils, which was left alone for a few minutes at a room temperature of 23 degrees and a humidity of 46%, after which the moisture content (corneometer) and sebum content (sebumeter) of the cheeks were measured. These results are given in Table 4.

TABLE 4
Aroma that gives a warm sensation

| | No aroma | Vanillin |
|---|---|---|
| Sebum content | no difference attributable to aroma | |
| Moisture content | 66.2 | 69.3 (increase) |

Aroma that gives a cold sensation

| | No aroma | Peppermint |
|---|---|---|
| Sebum content | 29.7 | 21.8 (decrease) |

| | |
|---|---|
| Moisture content | no difference attributable to aroma |

Working Examples 1 to 9 will now be given, which are specific examples of cosmetics in which the present invention is applied in an attempt to change usability, skin feel and so forth. The specific compositional components of the cold-sensation perfumes 1 to 4 and warm-sensation perfumes 1 to 4 added to the cosmetics in Working Examples 1 to 9, and the proportions in which these components were contained, are listed below.

### Cold-sensation Perfume 1

| Components | Amount contained |
|---|---|
| Dipropylene glycol | 3.7 |
| Dihydromyrcenol | 200 |
| Borneol | 1 |
| Precyclemon B (IFF) | 15 |
| Basil oil | 1 |
| Dynascone 10 | 2 |
| Rosemary oil | 130 |
| Juniper berry oil | 130 |
| Peppermint oil | 300 |
| Rose oxide | 0.3 |
| Mandarinal | 1 |
| Ambroxan | 15 |
| Cyclogalbanate | 20 |
| Triplal | 2 |
| Dimetol | 5 |
| Lemon oil | 40 |
| Clary sage oil | 2 |
| Galbanum oil | 2 |
| Lavender oil | 25 |
| Bergamot oil | 100 |
| Hexyl salicylate | 5 |
| Total | 1000 |

Cold-sensation perfume 2

| Components | Amount contained |
|---|---|
| Dipropylene glycol | 8 |
| Menthone | 50 |
| Isomenthone | 50 |
| Shinus molle oil | 10 |
| Rosemary oil | 300 |
| Cardamon oil | 2 |
| Juniper berry oil | 130 |
| Peppermint oil | 400 |
| Lime distilled oil | 50 |
| Total | 1000 |

### Cold-sensation Perfume 3

| Components | Amount contained |
|---|---|
| Ambroxan | 15 |
| Amyl salicylate | 3 |
| Basil oil | 1 |
| Bergamot oil | 80 |
| Clary sage oil | 2 |
| Cypress oil | 10 |
| Damascone alpha | 2 |
| Dihydromyrcenol | 200 |
| 2,6-dimethylheptanol (dimetol (GIV)) | 5 |
| Dynascone 10 (FIR) | 1 |
| Evernyl (GIV) | 5 |
| Galbanum oil | 0.7 |
| Geranium oil | 10 |
| Hedione | 60 |
| Helional | 1 |
| Hexyl acetate | 1 |
| Triplal | 1 |
| Hydroxycitronellal | 10 |
| Iso-E-Super | 30 |
| Isoborneol | 1 |
| Isogalbanate | 20 |
| Juniperberry oil | 10 |
| Lavandin oil | 30 |
| Lemon oil | 30 |
| Vertfix coeur (IFF) | 100 |
| Linalool | 25 |
| Linalyl acetate | 100 |
| Lyral | 10 |
| Mandarin aldehyde | 0.5 |
| Mandarin oil | 5 |
| Menthol | 10 |
| Methyl anthranilate | 0.1 |
| Methyl ionone gamma | 15 |
| Methyl naphthyl ketone beta | 1 |
| Nerolin bromilia | 1 |
| Peppermint oil America rect. | 100 |
| Pine needle oil | 2 |
| Precyclemon b (IFF) | 10 |
| Rose oxide L | 0.1 |
| Rosemary oil | 5 |
| Sandalore | 2 |
| Sandranol | 1 |
| Tonalide | 20 |
| Dipropylene glycol | 15 |
| Floralozone (IFF) | 1 |
| Meronal (GIV) | 0.5 |
| Calone | 3 |
| Cis-3-hexenyl acetate | 1 |
| Cis-3-hexenol | 1 |
| 1,8-cineol | 30 |
| Camphor | 5 |
| Methyl pamplemousse | 2 |
| Peonile | 0.1 |
| Allyl amyl glycolate | 5 |
| Total | 1000 |

### Cold-sensation Perfume 4

| Components | Amount contained |
|---|---|
| Methy dihydrojasmonate | 300 |
| Lilial | 50 |
| Galaxolide (50% BB) | 50 |
| Linalool | 50 |
| Menthol | 200 |
| Linalyl acetate | 50 |
| Ambroxan(10% DPG) | 20 |
| Peppermint | 60 |
| Green base | 10 |
| Helional | 10 |
| Triethyl citrate | 100 |
| Bergamot | 100 |
| Total | 1000 |

### Warm-sensation Perfume 1

| Components | Amount contained |
|---|---|
| Dipropylene glycol | 89.4 |
| Hedione | 300 |
| Hexyl cinnamic aldehyde | 50 |
| Lilial | 30 |
| Iso-E-Super | 100 |
| Galaxolide | 30 |
| Indole | 0.2 |
| Phenyl ethyl alcohol | 35 |
| Phenylacetaldehyde | 0.1 |
| Beta-ionone | 20 |
| Eugenol | 3 |
| Florosa (Quest) | 50 |
| Givescone (GIV) | 1 |
| Tagete oil | 1 |
| Basil oil | 5 |
| Prunella (FIR) | 1 |
| Ambroxan | 5 |
| Bacdanol (IFF) | 10 |
| Undecalactone gamma | 0.2 |
| Jasmal (IFF) | 10 |
| Ethyl linalool | 100 |
| Sandalore (GIV) | 60 |
| Orange oil | 5 |
| Manzanate (Quest) | 0.1 |
| Ethyl vanillin | 2 |
| Cyclopentadecanone | 2 |
| Citronellol | 30 |
| Geraniol | 25 |
| Geranyl acetate | 10 |
| Vanillin | 10 |
| Methyl ionoe gamma | 15 |
| Total | 1000 |

Warm-sensation perfume 2

| Components | Amount contained |
|---|---|
| Orange oil | 15 |
| Linalyl acetate | 10 |
| Ethyl linalool | 35 |
| Styrallyl acetate | 1 |
| Citronellol | 10 |
| Florosa (Quest) | 20 |
| Nerol | 2 |
| DPG | [sic] |
| Methyl ionone gamma | 25 |
| Alpha-ionone | 2 |
| Geraniol | 7 |
| Cedryl methyl ether | 5 |
| Polysantol (FIR) | 15 |
| Iso-E-Super (IFF) | 200 |
| Lilial | 15 |
| Anbroxan | 10 |
| Bacdanol | 70 |
| Canthoxal | 10 |
| Heliotropine | 10 |
| Pentalide | 10 |
| Cis-3-hexenyl salicylate | 20 |
| Hedione | 250 |
| Habanolide | 25 |
| Galaxolide | 100 |
| Exaltenone (fir) | 15 |
| Ambrettolide | 3 |
| Coumarine | 5 |
| Lyral | 40 |
| Vanilline | 10 |
| Ethylene brassylate | 50 |
| Raspberry ketone | 10 |
| Total | 1000 |

Warm-sensation perfume 3

| Components | Amount contained |
|---|---|
| Hedione | 80 |
| Hexyl cinnamic aldehyde | 200 |
| Lilial | 30 |
| Indole | 0.5 |
| Linalool | 30 |
| Phenyl ethyl alcohol | 80 |
| Benzyl acetate | 120 |
| Linalyl acetate | 40 |
| Phenyl ethyl phenyl acetate | 4 |
| Polysantol (F) | 20 |
| Phenylethyl acetate | 2 |
| Phenylethyl isobutyrate | 2 |
| Benzyl benzoate | 40 |
| Dimthyl phenylethyl carbinol | 20 |
| Vertenex (IFF) | 2 |
| Helional (IFF) | 60 |
| Vertofix coeur (IFF) | 30 |
| Isobutyl quinoline | 0.5 |
| Heliotropine | 3 |
| Geranium oil | 10 |
| Patchouli oil | 10 |
| Cyclamen aldehyde | 2 |
| Ethyl vanillin | 10 |
| Gamma-decalactone | 20 |
| Ylang ylang oil #2 | 4 |
| Benzyl alcohol | 4 |
| Citronellol | 30 |
| Geraniol | 20 |
| Geranyl acetate | 20 |
| Methyl anthranilate | 40 |
| Evernyl (GIV) | 4 |
| Methyl ionone gamma | 20 |
| Cedar wood oil | 20 |
| Cinnamic alcohol | 10 |
| Phenyl ethyl salicylate | 2 |
| Sandalwood oil | 10 |
| Total | 1000 |

### Warm-sensation Perfume 4

| Components | Amount contained |
|---|---|
| Methyl dihydrojasmonate | 200 |
| Iso-E-Super | 100 |
| Galaxolide (50% BB) | 200 |
| Linalool | 30 |
| Linalyl acetate | 20 |
| Beta-ionone | 50 |
| Orange oil | 30 |
| Milk lactone | 100 |
| Ethyl acetate(10% DPG) | 20 |
| Ald. C₁₂ kauric (50% BB) | 5 |
| Triethyl citrate | 125 |
| Ethyl vanillin | 20 |
| ak moss | 100 |
| Total | 1000 |

### Working Example 1

Astringent lotion

| | |
|---|---|
| Ethanol | 40 |
| Dipropylene glycol | 1 |
| Polyoxyethylene polyoxypropylene | 0.1 |
| decyl tetradecyl ether | |
| Cinnamic anhydride | 1 |
| Salicylic acid | 0.1 |
| Sodium citrate | 0.2 |
| Zinc paraphenolsulfonate | 0.2 |
| Dipotassium glycyrrizinate | 0.1 |
| Pyridoxine hydrochloride | 0.1 |
| L-menthol | 0.05 |
| Trisodium HEDTA | 0.05 |
| Cellulose powder | 1 |
| Bentonite | 0.8 |
| Cold-sensation perfume 1 | 0.002 |
| Purified water | balance |

### Working Example 2

Sun block cream

| | |
|---|---|
| Decamethylcyclopentanesiloxane | 20 |
| Trimethylsiloxysilicic acid | 1 |
| Polyoxyethylene-methylpolysiloxane copolymer | 2 |
| Dipropylene glycol | 4 |
| Squalane | 5 |
| Silicone-covered titanium oxide | 10 |
| microparticles | |
| Talc (hydrophobically treated) | 6 |
| Paraben | appropriate amount |
| Phenoxyethanol | appropriate amount |
| Trisodium edetate | 0.02 |
| 4-t-butyl-4′-methoxydibenzoylmethane | 0.1 |
| 2-ethylhexyl p-methoxycinnamate | 7 |
| Glyceryl mono-2-ethylhexanoate di-p- | 0.5 |
| methoxycinnamate | |
| Spherical polyethylene powder | 5 |
| Dimethyldistearylammonium hectorite | 1 |
| Cold-sensation perfume 2 | 0.05 |
| Purified water | balance |

### Working Example 3

Gelled aromatic

| A. Main aqueous part | |
|---|---|
| Gellan gum | 1 |
| edetate | 0.05 |
| water | balance |
| B. Electrolyte part | |
| Water | 10 |
| Triethylamine hydrochloride | 1 |
| C. Alcohol part | |
| Ethanol | 10 |
| Phenoxyethanol | 0.3 |
| Polyoxyethylene hydrogenated castor oil | 5 |
| Polyoxyethylene octyldodecyl ether | 5 |
| Cold-sensation perfume 2 | 5 |
| Bitterness agent (desodium benzoate) | 0.005 |

### Manufacturing Method

Part A was heated and dissolved at 80° C., after which part B was added and the system was cooled. At the point when the system reached 60° C., the uniformly dissolved part C was added under stirring, and the solution thus obtained was poured into a vessel and then allowed to stand, cool to room temperature, and solidify, which gave a gelled aromatic.

### Working Example 4

Body soap

| | |
|---|---|
| Propylene glycol | 2.0 |
| Ethylene glycol distearate | 2.0 |
| Coconut oil fatty acid diethanolamide | 6.0 |
| Sodium dodecan-1,2-diol acetic acid ether | 2.0 |
| Polyoxyethylene lauryl ether sodium sulfate | 7.5 |
| Polyoxyethylene lauryl ether sulfuric acid | 3.0 |
| triethanolamine | |
| Coconut oil fatty acid methyltaurine sodium | 3.0 |
| Coconut oil fatty acid amide propyl betaine | 1.0 |
| Citric acid (50% solution) | 0.5 |
| Sodium citrate | 1.0 |
| Sodium benzoate | appropriate amount |
| Disodium edetate | appropriate amount |
| Cold-sensation perfume 3 | 0.5 |
| Pure water | balance |

### Working Example 5

Antiperspirant aerosol cosmetic

| A. Powder component | | |
|---|---|---|
| Aluminum hydroxychloride | 3 | mass % |
| Zinc oxide | 2 | |
| Silica | 3 | |
| Cornstarch | 0.1 | |
| Calcium stearate | 0.1 | |
| B. Oil phase component | | |
| Dimethylpolysiloxane | 2 | |
| Cetyl octane | 2 | |
| POE (10) POP (10) dimethyl ether (random | 10 | |
| copolymer) | | |
| Sorbitan oleate | 1 | |
| Antioxidant | appropriate amount | |
| Cold-sensation perfume 4 | 0.05 | |
| C. Propellant | | |

| | |
|---|---|
| Liquefied petroleum gas | balance |

### Manufacturing Method and Assessment

The powder (A) and the oil phase component (B) were mixed, and the propellant (C) was charged into this mixture, which gave an antiperspirant aerosol cosmetic.

### Working Example 6

Emulsion

| | |
|---|---|
| Dimethylpolysiloxane | 2 |
| Behenyl alcohol | 1 |
| Batyl alcohol | 0.5 |
| Glycerin | 5 |
| 1,3-butylene glycol | 7 |
| Erythritol | 2 |
| Hydrogenated oil | 3 |
| Squalane | 6 |
| Tetra-2-ethylhexanoic acid pentaerythritol | 2 |
| Polyoxyethylene glyceryl isostearate | 1 |
| Polyoxyethylene glycerin monostearate | 1 |
| Potassium hydroxide | appropriate amount |
| Sodium hexametaphosphate | 0.05 |
| Phenoxyethanol | appropriate amount |
| Carboxyvinyl polymer | 0.1 |
| Warm-sensation perfume 1 | 0.05 |
| Purified water | balance |

### Working Example 7

Moisturizing cream

| | |
|---|---|
| Liquid paraffin | 10 |
| Dimethylpolysiloxane | 2 |
| Glycerin | 10 |
| 1,3-butylene glycol | 2 |
| Erythritol | 1 |
| Polyethylene glycol 1500 | 5 |
| Squalane | 15 |
| Tetra-2-ethylhexanoic acid pentaerythritol | 5 |
| Potassium hydroxide | 0.1 |
| Sodium hexametaphosphate | 0.05 |
| Tocopherol acetate | 0.05 |
| p-Hydroxybenzoic acid ester | appropriate amount |
| Hydroxypropyl methyl cellulose | 0.3 |
| Polyvinyl alcohol | 0.1 |
| Carboxyvinyl polymer | 0.2 |
| Acrylic acid-alkyl methacrylate copolymer | 0.1 |
| (Pemulen TR-2) | |
| Warm-sensation perfume 2 | 0.03 |
| Purified water | balance |

### Working Example 8

Foundation

| | |
|---|---|
| Dimethylpolysiloxane | 15 |
| Decamethylcyclopentanesiloxane | 20 |
| Polyoxyethylene-methylpolysiloxane copolymer | 5 |
| High-molecular weight amino-modified silicone | 0.1 |
| Glycerin | 5 |
| 1,3-butylene glycol | 10 |
| Palmitic acid | 0.5 |
| Macadamia nut oil fatty acid cholesterol | 0.1 |
| Distearyldimethylammonium chloride | 0.2 |
| Alkyl-modified silicon resin-covered yellow | 2 |
| iron oxide | |
| Alkyl-modified silicon resin-covered red | 1 |
| iron oxide | |
| Alkyl-modified silicon resin-covered black | 0.3 |
| iron oxide | |
| Alkyl-modified silicon resin-covered | 10 |
| titanium oxide | |
| Alkyl-modified silicon resin-covered talc | 1.5 |
| oxide | |
| Silicone-covered fusiform titanium oxide | 3 |
| Sodium L-glutamate | 0.5 |
| DL-α-tocopherol acetate | 0.1 |
| p-Hydroxybenzoic acid ester | appropriate amount |
| Methylbis(trimethylsiloxy)silylisopentyl | 0.1 |
| trimethoxycinnamate | |
| Dimethyldistearylammonium hectorite | 1.5 |
| Spherical nylon powder | 1 |
| Warm-sensation perfume 3 | 0.02 |
| Purified water | balance |

### Working Example 9

Gelled aromatic

| | |
|---|---|
| Dipropylene glycol | 5 |
| Propylene glycol | 5 |
| POE 60 hydrogenated castor oil | 4 |
| POE 15 lauryl ether | 4 |
| Kappa type carrageenan | 1.4 |
| Iota type carrageenan | 0.6 |
| Hydrophobic modified polyether urethane | 2 |
| (Adekanol GT-700) | |
| pH regulator: sodium citrate | 0.5 |
| Warm-sensation perfume 4 | 5 |
| Purified water | balance |

### Working Example 10

Hair shampoo

| | |
|---|---|
| Cation-modified locust bean gum*¹ | 0.3 |
| Cation-modified fenugreek gum*^{2 [4]} | 0.2 |
| Coconut oil fatty acid amide propyl betaine | 5 |
| POE (2) lauryl ether sodium sulfate | 4.5 |
| Propylene glycol laurate | 2.1 |
| N-cocoyl-N-methyltaurine-N′-methyltaurine | 6 |
| sodium | |
| Ethylene glycol distearate | 1.5 |
| Oleic acid monoglyceride | 0.1 |
| Silicone emulsion*³ | 1.5 |
| Sodium benzoate | 0.3 |
| Phenoxyethanol | 0.1 |
| Warm-sensation perfume 3 | 0.03 |
| EDTA-2Na•2H₂O | 0.05 |
| Tap water | balance |

*¹Catinal CLB-100 (made by Toho Chemical)
*²Catinal CF-100 (made by Toho Chemical)
*³Dimethylsilicone emulsion BY22-007 (containing 50 mass % dimethylpolysiloxane; made by Toray-Dow Corning)

### Working Example 11

Hair rinse, treatment

| | |
|---|---|
| High-polymerization dimethylsiloxane- | 0.2 |
| methyl(aminopropyl)siloxane copolymer | |
| Hydrogenated rape oil alcohol | 3 |
| Glycerin | 3.5 |
| 3-methyl-1,3-butanediol | 5 |
| Hydroxystearic acid | 0.5 |
| Cetyl 2-ethylhexanoate | 1 |
| Isononyl isononanate | 0.5 |
| Sensomer CI-50 (made by Nalco) | 0.2 |
| Stearic acid dimethylaminopropylamide | 1 |
| Merquat 550 (made by Calgon) | 1 |
| L-glutamic acid | 0.5 |
| Phenoxyethanol | 0.5 |
| Lecithin | 0.1 |
| Cold-sensation perfume^{[5]} | 0.05 |
| Pure water | balance |
| Coloring | appropriate amount |

### FIG. 1

Without aroma unscented cream (model composition).
cotton (unscented)
With aroma
cotton (scented)
unscented cream (model composition)

### FIG. 2

Relationship Between Cream Temperature Sense Brought about by Aroma and the “Freshness of The Skin”

Cream temperature sense
Makes skin feel fresh

Makes skin feel somewhat fresh Can't say either way

Does not make skin feel very fresh
Does not make skin feel fresh

### Cold

Somewhat cold
Can't say either way

Somewhat warm

### Warm

FIG. 3

Relationship between cream temperature sense brought about by aroma and the “moistness of the skin”

### Cream Temperature Sense

Makes skin feel moist
Makes skin feel somewhat moist
Can't say either way
Does not make skin feel very moist
Does not make skin feel moist

### Cold

Somewhat cold
Can't say either way
Somewhat warm

### Warm

### FIG. 4

### Natural

Rich (feminine)

### Sharp

### Tender

light spreading
   transparent
   bright fresh
bracing
   moist
   mild
   sweet
      heavy spreading
   sultry
   thick

### FIG. 5

1. very warm
2. warm
3. somewhat warm
4. can't say either way
5. somewhat cold
6. cold
7. very cold

### FIG. 7

Sense of warmth or cold inside the box

### Peppermint

### Bergamot

### Vanillin

1. very warm
2. warm
3. somewhat warm
4. can't say either way (same as with no aroma)
5. somewhat cold
6. cold
7. very cold

FIG. 8

Aroma intensity inside the box

### Peppermint

### Bergamot

### Vanillin

1. Do not sense aroma
2. Sense aroma, but faintly
3. Aroma just the right strength
4. Aroma too strong

1: A method for controlling temperature sense or skin feel comprising:
(a) preparing a perfume composition for temperature sense control including at least one perfume component selected from the identified group of the perfume component after identifying the group of a perfume component that raises the temperature subjectively sensed (temperature sense) when a person smells the perfume component by examining the change in temperature sense under the condition both with and without aroma, and
(b) applying an article to the human skin while having a person smell the perfume composition for temperature sense control including the at least one selected perfume component, wherein
at least one of temperature sense and skin feel that the person holds to the article is changed, and, wherein
the identified group of the perfume component consists of vanillin, 4-tert-butyl-α-methylhydrocinnamic aldehyde, heliotropine, 4,6,6,7,8,8-hexamethyl-1,3,4,6,7,8-hexahydrocyclopentabenzopyran, γ-undecalactone, β-ionone, cumin oil, lavender oil, clove oil, 3α,6,6,9α-tetramethyldodecahydronaphtho[2,1-b]furan, and maltol.

2: A method for controlling temperature sense or skin feel comprising:
(a) preparing a perfume composition for temperature sense control including at least one perfume component selected from the identified group of the perfume component after identifying the group of a perfume component that lowers the temperature subjectively sensed (temperature sense) when a person smells the perfume component by examining the change in temperature sense under the condition both with and without aroma, and
(b) applying an article to the human skin while having a person smell the perfume composition for temperature sense control including the at least one selected perfume component, wherein
at least one of temperature sense and skin feel that the person holds to the article is changed, and wherein
the identified group of the perfume component consists of bergamot oil, lime oil, 7-methyl-3,4-dihydro-(2H)-1,5-benzodioxepin-3-one, chamomile oil, 2,4-dimethyl-3-cyclohexenyl carboxyaldehyde, majolaine oil, patchouli oil, jasmine absolute, sandalwood oil, geranium oil, rose oil, and methyl-N-3,7-dimethyl-7-hydroxyoctylidene-anthranilate.

3: The method for controlling temperature sense or skin feel according to claim 1, wherein the article is a cosmetic.

4: The method for controlling temperature sense or skin feel according to claim 2, wherein the article is a cosmetic.

5: A method for controlling temperature sense or skin feel comprising:
(a) preparing a perfume composition for temperature sense control including at least one perfume component selected from the identified group of the perfume component after identifying the group of a perfume component that raises the temperature subjectively sensed (temperature sense) when a person smells the perfume component by examining the change in temperature sense under the condition both with and without aroma, and
(b) applying an article compounding the perfume composition for temperature sense control to the human skin while having a person smell the perfume composition for temperature sense control including the at least one selected perfume component, wherein
at least one of temperature sense and skin feel that the person holds to the article is changed, and wherein
the identified group of the perfume component consists of vanillin, 4-tert-butyl-α-methylhydrocinnamic aldehyde, heliotropine, 4,6,6,7,8,8-hexamethyl-1,3,4,6,7,8-hexahydrocyclopentabenzopyran, γ-undecalactone, β-ionone, cumin oil, lavender oil, clove oil, 3α,6,6,9° α-tetramethyldodecahydronaphtho[2,1-b]furan, and maltol.

6: A method for controlling temperature sense or skin feel comprising:
(a) preparing a perfume composition for temperature sense control including at least one perfume component selected from the identified group of the perfume component after identifying the group of a perfume component that lowers the temperature subjectively sensed (temperature sense) when a person smells the perfume component by examining the change in temperature sense under the condition both with and without aroma, and
(b) applying an article compounding the perfume composition for temperature sense control to the human skin while having a person smell the perfume composition for temperature sense control including the at least one selected perfume component, wherein
at least one of temperature sense and skin feel that the person holds to the article is changed, and wherein
the identified group of the perfume component consists of bergamot oil, lime oil, 7-methyl-3,4-dihydro-(2H)-1,5-benzodioxepin-3-one, chamomile oil, 2,4-dimethyl-3-cyclohexenyl carboxyaldehyde, majolaine oil, patchouli oil, jasmine absolute, sandalwood oil, geranium oil, rose oil, and methyl-N-3,7-dimethyl-7-hydroxyoctylidene-anthranilate.

7: The method for controlling temperature sense or skin feel according to claim 5, wherein the article is a cosmetic.

8: The method for controlling temperature sense or skin feel according to claim 6, wherein the article is a cosmetic.

9: A method of increasing skin moisture content, comprising having a person smell the perfume component or perfume composition according to claim 1.

10: A method of increasing skin moisture content, comprising having a person smell the perfume component or perfume composition according to claim 5.

11: A method of reducing skin sebum content, comprising having a person smell the perfume component or perfume composition according to claim 2.

12: A method of reducing skin sebum content, comprising having a person smell the perfume component or perfume composition according to claim 6.
